(19) United States
(12) Patent Application Publication
(10) Pub. No.: US 2025/0188443 A1
(43) Pub. Date: Jun. 12, 2025

(54) METHOD FOR CULTIVATING CYANOBACTERIA

(71) Applicant: NAN YA PLASTICS CORPORATION, TAIPEI (TW)

(72) Inventors: TE-CHAO LIAO, TAIPEI (TW); JUNG-JEN CHUANG, TAIPEI (TW); YI-CHENG LI, TAIPEI (TW); Tzu-Huan Wong, Hualien County (TW); Liang-Jung Chien, TAIPEI (TW)

(21) Appl. No.: 18/427,722

(22) Filed: Jan. 30, 2024

(30) Foreign Application Priority Data
Dec. 11, 2023 (TW) 112148039

(51) Int. Cl. C12N 15/10; C12N 1/20

(52) U.S. Cl. CPC C12N 15/1058; C12N 1/20

(57) ABSTRACT

A method for cultivating cyanobacteria includes: providing the cyanobacteria; subjecting the cyanobacteria to physical mutagenesis, so as to obtain primary mutant cyanobacteria; subjecting the primary mutant cyanobacteria to chemical mutagenesis, so as to obtain secondary mutant cyanobacteria; and subjecting the secondary mutant cyanobacteria to a temperature resistance test and an environment resistance test, so as to obtain target cyanobacteria. A fatality rate of the physical mutagenesis ranges between 50% and 80%, and a fatality rate of the chemical mutagenesis ranges between 40% and 55%.

### CROSS-REFERENCE TO RELATED PATENT APPLICATION

This application claims the benefit of priority to Taiwan Patent Application No. 112148039, filed on Dec. 11, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for cultivating cyanobacteria, and more particularly to a method for cultivating cyanobacteria that are capable of withstanding a high-temperature industrial waste gas.

### BACKGROUND OF THE DISCLOSURE

Cyanobacteria are commonly referred to as blue-green algae, which is a prokaryote that is capable of photosynthesis. In the conventional technology, the photosynthetic property of the cyanobacteria can be utilized to absorb carbon dioxide discharged by factories, which aims to achieve a cycle of sustainable development with zero waste and zero pollution.

However, in addition to highly-concentrated carbon dioxide, an industrial waste gas further includes sulfide, nitride, and other chemical substances. These chemical substances usually affect growth of the cyanobacteria, and may even result in death of the cyanobacteria. Furthermore, the temperature of the industrial waste gas is often high, such that a survival rate of the cyanobacteria is significantly decreased, and a photosynthetic efficiency is also significantly decreased. Hence, a carbon fixation property of the cyanobacteria cannot be easily and effectively applied to processing of the industrial waste gas.

Therefore, how to cultivate cyanobacteria that are capable of withstanding a high temperature and an industrial waste gas, so as to overcome the above-mentioned deficiencies, has become one of the important issues to be solved in the relevant industry.

### SUMMARY OF THE DISCLOSURE

In response to the above-referenced technical inadequacies, the present disclosure provides a method for cultivating cyanobacteria.

In order to solve the above-mentioned problems, one of the technical aspects adopted by the present disclosure is to provide a method for cultivating cyanobacteria. The method includes: providing the cyanobacteria; subjecting the cyanobacteria to physical mutagenesis, so as to obtain primary mutant cyanobacteria; subjecting the primary mutant cyanobacteria to chemical mutagenesis, so as to obtain secondary mutant cyanobacteria; and subjecting the secondary mutant cyanobacteria to a temperature resistance test and an environment resistance test, so as to obtain target cyanobacteria. A fatality rate of the physical mutagenesis ranges between 50% and 80%, and a fatality rate of the chemical mutagenesis ranges between 40% and 55%.

In one of the possible or preferred embodiments, the fatality rate of the physical mutagenesis ranges between 60% and 80%.

In one of the possible or preferred embodiments, the fatality rate of the chemical mutagenesis ranges between 45% and 55%.

In one of the possible or preferred embodiments, an ultraviolet (UV) source having an illuminance of between 0.017 mW/cm² and 0.082 mW/cm² is used in the physical mutagenesis to irradiate the cyanobacteria for 10 seconds to 70 seconds.

In one of the possible or preferred embodiments, nitrosoguanidine (NTG) having a concentration of between 50 μg/ml and 300 μg/ml is used in the chemical mutagenesis for a processing time within a range between 0.5 minutes and 2 minutes.

In one of the possible or preferred embodiments, the secondary mutant cyanobacteria are placed in an environment being within a range between 30° C. and 60° C. for monitoring a growth status in the temperature resistance test, so as to filter out initial cyanobacteria.

In one of the possible or preferred embodiments, the initial cyanobacteria are placed in a mixed gas at a ventilation ratio within a range between 0.5% and 9% in the environment resistance test, so as to filter out the target cyanobacteria.

In one of the possible or preferred embodiments, the mixed gas includes hydrogen, acetylene, methane, hydrogen sulfide, and acetaldehyde.

In one of the possible or preferred embodiments, the mixed gas includes 30 ppm to 50 ppm of the hydrogen, 150 ppm to 250 ppm of the acetylene, 100 ppm to 200 ppm of the methane, 0.1 ppm to 1 ppm of the hydrogen sulfide, and 1 ppm to 5 ppm of the acetaldehyde.

In one of the possible or preferred embodiments, the method further includes: subjecting the secondary mutant cyanobacteria to the chemical mutagenesis again.

In order to solve the above-mentioned problems, another one of the technical aspects adopted by the present disclosure is to provide a method for cultivating cyanobacteria. The method includes: providing the cyanobacteria; subjecting the cyanobacteria to physical mutagenesis until a fatality rate reaches 50% to 80%, so as to obtain and use the cyanobacteria that survive the physical mutagenesis as primary mutant cyanobacteria; subjecting the primary mutant cyanobacteria to chemical mutagenesis until the fatality rate reaches 40% to 55%, so as to obtain and use the primary mutant cyanobacteria that survive the chemical mutagenesis as secondary mutant cyanobacteria; placing the secondary mutant cyanobacteria in an environment being within a range between 30° C. and 60° C. for monitoring a growth status, so as to filter out initial cyanobacteria; and placing the initial cyanobacteria in a mixed gas at a ventilation ratio within a range between 0.5% and 9%, so as to filter out target cyanobacteria. The mixed gas is a mixture that includes hydrogen, acetylene, methane, hydrogen sulfide, and acetaldehyde.

In one of the possible or preferred embodiments, an ultraviolet (UV) source having an illuminance of between 0.017 mW/cm² and 0.082 mW/cm² is used in the physical mutagenesis to irradiate the cyanobacteria for 10 seconds to 70 seconds.

In one of the possible or preferred embodiments, nitrosoguanidine (NTG) having a concentration of between 50 μg/ml and 300 μg/ml is used in the chemical mutagenesis for a processing time within a range between 0.5 minutes and 2 minutes.

In one of the possible or preferred embodiments, the mixed gas includes 30 ppm to 50 ppm of the hydrogen, 150 ppm to 250 ppm of the acetylene, 100 ppm to 200 ppm of the methane, 0.1 ppm to 1 ppm of the hydrogen sulfide, and 1 ppm to 5 ppm of the acetaldehyde.

In one of the possible or preferred embodiments, the method further includes: subjecting the secondary mutant cyanobacteria to the chemical mutagenesis again.

Therefore, in the method for cultivating the cyanobacteria provided by the present disclosure, by virtue of “subjecting the cyanobacteria to the physical mutagenesis and the chemical mutagenesis” and “the fatality rate of the physical mutagenesis ranging between 50% and 80%, and the fatality rate of the chemical mutagenesis ranging between 40% and 55%,” the probability of obtaining cyanobacteria that are capable of withstanding a high-temperature industrial waste gas can be increased.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a method for cultivating cyanobacteria according to a first embodiment of the present disclosure;

FIG. 2 is a curve diagram showing influence of a height and time of ultraviolet (UV) irradiation upon a fatality rate of a strain;

FIG. 3 is a schematic diagram showing ratios that a strain being capable of withstanding a temperature of 45° C. or more can be filtered out through altering ultraviolet (UV) conditions;

FIG. 4 is a curve diagram showing influence of a nitrosoguanidine (NTG) concentration and a processing time upon the fatality rate of the strain;

FIG. 5 is a schematic diagram showing ratios that the strain being capable of withstanding a temperature of 45° C. or more can be filtered out through altering nitrosoguanidine (NTG) conditions;

FIG. 6 is a flowchart of the method for cultivating the cyanobacteria according to a second embodiment of the present disclosure; and

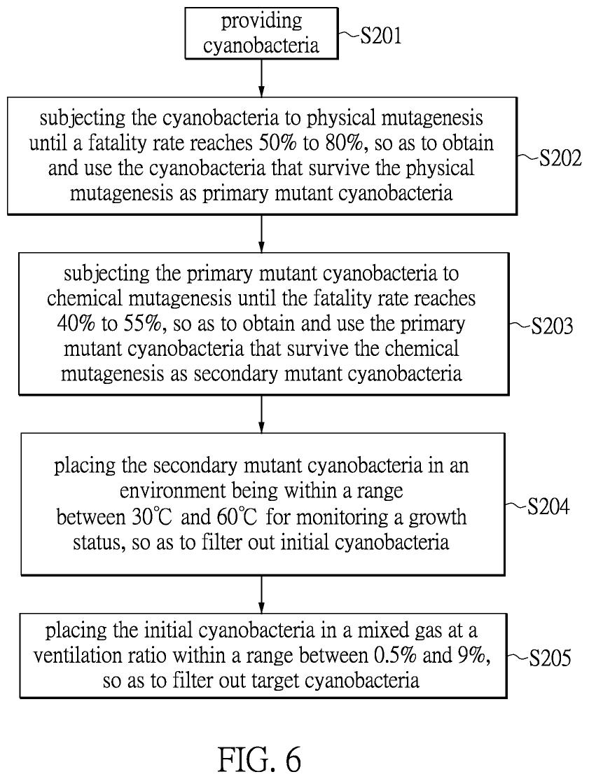

FIG. 7 is a schematic diagram showing carbon dioxide (CO₂) consumption of the cyanobacteria that is filtered out by the method of the present disclosure when processing industrial waste gas.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

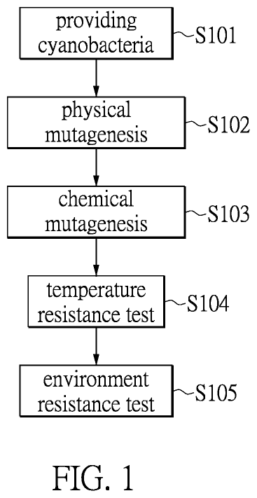

Referring to FIG. 1, a method for cultivating cyanobacteria provided in the first embodiment of the present disclosure includes providing the cyanobacteria (step S101). In one embodiment of the present disclosure, the cyanobacteria of the present disclosure are Synechococcus elongatus PCC7942. In the present disclosure, the cyanobacteria are subjected to physical mutagenesis (step S102), and are then subjected to chemical mutagenesis (step S103), so as to effectively obtain cyanobacteria that are capable of withstanding a high temperature and an industrial waste gas.

In the physical mutagenesis (step S102), the cyanobacteria (with use of a BG-11 solid medium) are firstly placed and cultivated in an incubator having a CO₂ concentration of 3% for about 36 hours at 30° C. Then, a strain is quantitated at 10⁵ cells/mL by a cell counter (e.g., a SCEPTER™ handheld automated cell counter produced by Millipore). The strain is placed under a UV light source for the physical mutagenesis. After reactions, a bacterial suspension is spread on a petri dish of 10 cm×10 cm, and is grown in the incubator at 30° C. A sterilized toothpick is used to pick and move a grown colony to a new BG-11 solid medium for preservation and subsequent analysis.

The UV light source of the present disclosure can be ALL KILL-01 (which is a purifier for sanitization and sterilization, and has ultraviolet germicidal irradiation (UVGI), 253.7 nm of UV-C, and 20,000 μw-sec/cm² of luminous energy) produced by Power Jade Link Energy Technology Inc. A fatality rate of the physical mutagenesis (step S102) needs to be controlled between 50% and 80% (preferably between 60% and 80%, and more preferably 75%), so as to facilitate subsequent formation of cyanobacteria that are capable of withstanding a high temperature and an industrial waste gas. When the fatality rate is less than 50% or greater than 80%, the probability of obtaining high-temperature-resistant and environment-resistant cyanobacteria is decreased.

Specifically, the fatality rate of the present disclosure refers to dividing a growth quantity of the strain that is spread on a solid medium after a mutagenesis test by a growth quantity of the strain that is spread on the solid medium before the mutagenesis test and multiplying by 100%. The quantity of the strain before the mutagenesis test is calculated by the cell counter, and sterile water is added to produce a bacterial suspension quantitated at 105 cells/mL. After continuous dilution, the strain that has undergone the physical mutagenesis is spread on the BG-11 solid medium for calculating an actual quantity of the colony.

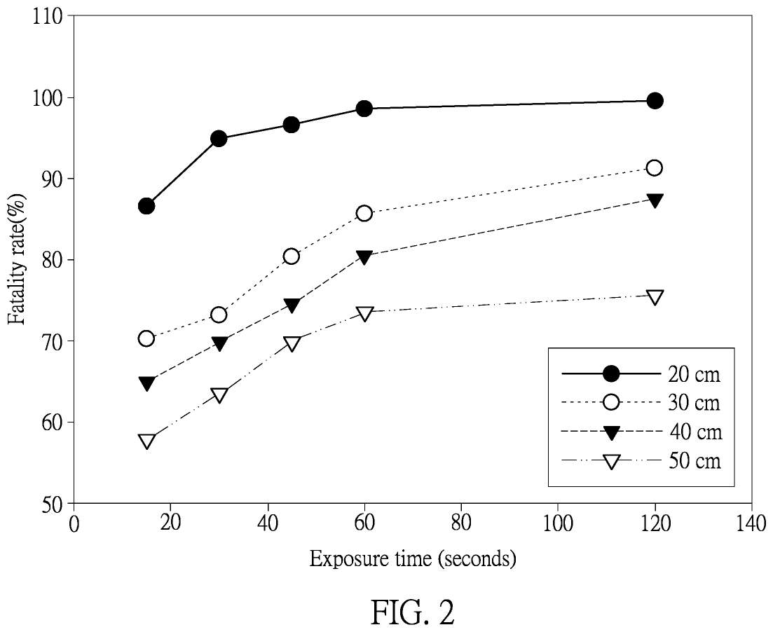

In the present disclosure, through a specific UV light source and a specific processing time, the fatality rate may fall within a specific range, and the high-temperature-resistant and environment-resistant cyanobacteria can be effectively obtained. In order to control the fatality rate of the physical mutagenesis (step S102) to range between 50% and 80%, how a fatality rate of the strain is influenced by different heights and different lengths of processing time of the UV light source is further analyzed in the present disclosure. As shown in FIG. 2, when a vertical height between the UV light source and the strain is between 20 cm and 50 cm, and an exposure time is between 10 seconds and 70 seconds, the fatality rate of the strain can be controlled between 50% and 80%. In one exemplary embodiment of the present disclosure, when the vertical height between the UV light source and the strain is between 30 cm and 40 cm, and the exposure time is between 30 seconds and 40 seconds, the fatality rate of the strain can be controlled between 70% and 75%.

Reference can be made to Table 1 below, which illustrates a corresponding relationship between an illuminance received by the strain and the vertical height between the UV light source and the strain when the used UV light source is UV-C (253.7 nm).

TABLE 1

| Distance (cm) | Illuminance (mW/cm²) |
|---|---|
| 0 | 2.160 |
| 2.5 | 1.276 |
| 5 | 0.693 |
| 10 | 0.220 |
| 20 | 0.082 |
| 40 | 0.039 |
| 50 | 0.017 |
| 60 | 0.000 |

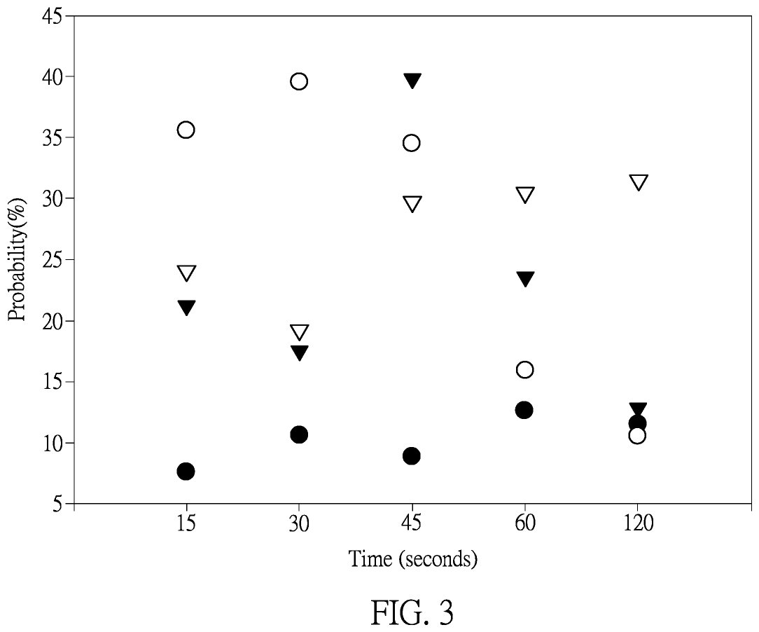

Reference can be further made to FIG. 3, which is a schematic diagram showing ratios that a strain being capable of withstanding a temperature of 45° C. or more can be filtered out through altering ultraviolet (UV) conditions. In FIG. 3, two points at which the probability of obtaining the strain that is capable of withstanding a temperature of 45° C. or more is the highest are respectively when the vertical height between the UV light source and the strain is 30 cm and an irradiation time is 30 seconds, and when the vertical height between the UV light source and the strain is 40 cm and the irradiation time is 45 seconds. The probability of filtering out a temperature-resistant strain under these conditions is approximately 41.5%. That is to say, in order to increase the probability of obtaining the strain that is capable of withstanding a temperature of 45° C. or more, the fatality rate of the physical mutagenesis (step S102) needs to be controlled between 50% and 80% (preferably between 60% and 80%, and more preferably 75%).

Then, the strain that survives the physical mutagenesis (step S102) is subjected to the chemical mutagenesis (step S103), and the chemical mutagenesis (step S103) of the present disclosure can be methylnitronitrosoguanidine (NTG) mutagenesis. That is, N-methyl-N′-nitro-N-nitrosoguanidine having a specific concentration is configured to induce mutagenesis of the strain.

Specifically, the strain (with use of the BG-11 solid medium) is placed and cultivated in the incubator at 30° C. for about 36 hours, and is then quantitated at 105 cells/mL by the cell counter. By adding the sterile water or a BG-11 medium, a bacterial suspension (4 ml) is produced and stored in a 30° C. water bath for use. In the present embodiment, an NTG reaction agent is made by adding 1.5 mg of nitrosoguanidine (NTG) into a sterilized centrifuge tube and adding 1 ml of a phosphate buffer solution (pH 6, 0.2 M) to dissolve the NTG, and is stored in the 30° C. water bath for use.

During the chemical mutagenesis (step S103), the bacterial suspension is poured and properly mixed in the centrifuge tube that contains the NTG reaction agent, and is immediately placed in the 30° C. water bath for calculating a reaction time. In the present embodiment, a final concentration of the NTG is 300 μg/ml. After the NTG reaction agent is reacted for a predetermined period of time, the centrifuge tube is taken out of the water bath, and is centrifuged for 10 minutes at 3,500 rpm. A waste liquid is poured into highly-concentrated sodium hydroxide (NaOH), a pellet is evenly mixed, and 5 ml of saline is added for centrifugation at 3,500 rpm for 10 minutes. By removing the waster liquid and further adding 5 ml of the sterile water, a bacterial suspension that has undergone the NTG mutagenesis can be produced. Afterwards, the bacterial suspension that has undergone the NTG mutagenesis is spread on the petri dish of 10 cm×10 cm, and is grown in the incubator at 25° C. The sterilized toothpick is used to pick and move the grown colony to the new BG-11 solid medium for preservation and subsequent analysis.

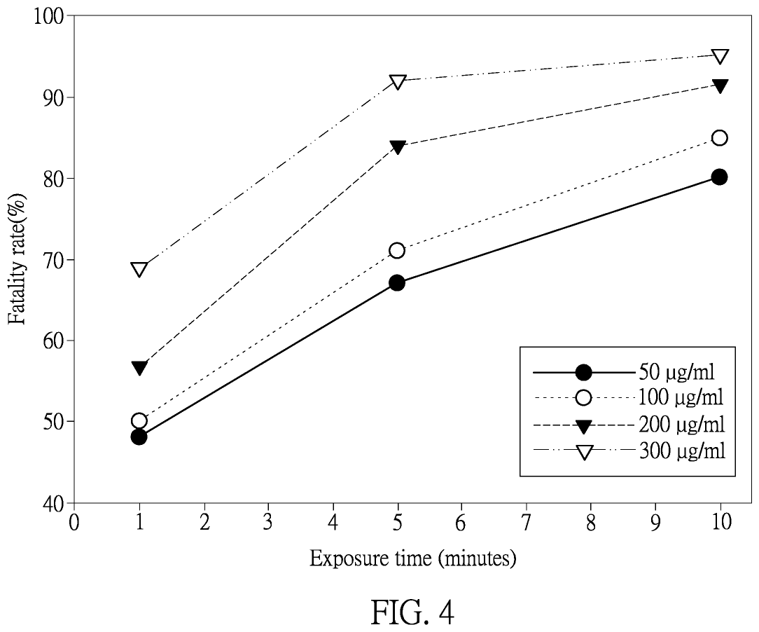

In the present disclosure, through a specific NTG concentration and a specific processing time, the fatality rate may fall within a specific range, and more high-temperature-resistant and environment-resistant cyanobacteria can be obtained. In order to control the fatality rate of the chemical mutagenesis (step S103) to range between 40% and 55%, how the fatality rate of the strain is influenced by different NTG concentrations and different lengths of processing time is further analyzed in the present disclosure. As shown in FIG. 4, the strain is processed for 0 minutes to 10 minutes by using 50 μg/ml to 300 μg/ml of the NTG. When 50 μg/ml to 200 μg/ml of the NTG is used to process the strain for 0.5 minutes to 2 minutes, the fatality rate of the strain can be controlled between 40% and 55%. In one exemplary embodiment of the present disclosure, when 50 μg/ml to 100 μg/ml of the NTG is used to process the strain for 0.5 minutes to 1.5 minutes, the fatality rate of the strain can be controlled to be approximately 50%.

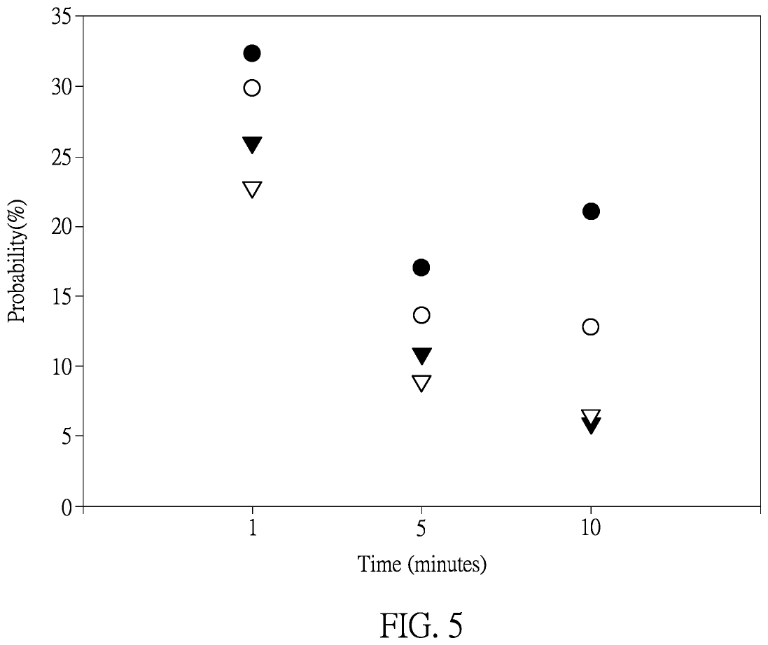

Reference can be further made to FIG. 5, which is a schematic diagram showing ratios that the strain being capable of withstanding a temperature of 45° C. or more can be filtered out through altering nitrosoguanidine (NTG) conditions. In FIG. 5, two points at which the probability of obtaining the strain that is capable of withstanding a temperature of 45° C. or more is the highest are respectively 50 μg/ml of the NTG and 100 μg/ml of the NTG (with the strain being processed for 1 minute). The probability of filtering out the temperature-resistant strain under these conditions ranges approximately from 30% to 33%. That is to say, in order to increase the probability of obtaining the strain that is capable of withstanding a temperature of 45° C. or more, the fatality rate of the chemical mutagenesis (step S103) needs to be controlled between 40% and 55% (preferably between 45% and 55%, and more preferably 50%).

The strain that survives the chemical mutagenesis (step S103) is then subjected to a temperature resistance test (step S104). In one embodiment of the present disclosure, the strain that is subjected to the temperature resistance test (step S104) is approximately 12.5% of the original cyanobacteria provided in step S101.

### Second Embodiment

In the second embodiment of the present disclosure, a method for cultivating cyanobacteria includes providing the cyanobacteria (step S201). In step S202, the cyanobacteria are subjected to physical mutagenesis until a fatality rate reaches 50% to 80%, and 20% to 50% of the cyanobacteria that survive the physical mutagenesis are used as primary mutant cyanobacteria. Preferably, the cyanobacteria are subjected to the physical mutagenesis in step S202 until the fatality rate reaches 60% to 80%, and 20% to 40% of the cyanobacteria that survive the physical mutagenesis are used as the primary mutant cyanobacteria. More preferably, the cyanobacteria are subjected to the physical mutagenesis in step S202 until the fatality rate reaches 75%, and 25% of the cyanobacteria that survive the physical mutagenesis are used as the primary mutant cyanobacteria.

In step S203, the primary mutant cyanobacteria are subjected to chemical mutagenesis until the fatality rate reaches 40% to 55%, and 45% to 60% of the primary mutant cyanobacteria that survive the chemical mutagenesis are used as secondary mutant cyanobacteria. Preferably, the primary mutant cyanobacteria are subjected to the chemical mutagenesis in step S203 until the fatality rate reaches 45% to 55%, and 55% to 60% of the primary mutant cyanobacteria that survive the chemical mutagenesis are used as the secondary mutant cyanobacteria. More preferably, the primary mutant cyanobacteria are subjected to the chemical mutagenesis in step S203 until the fatality rate reaches 50%, and 50% of the primary mutant cyanobacteria that survive the chemical mutagenesis are used as the secondary mutant cyanobacteria.

In step S204, the secondary mutant cyanobacteria are placed in an environment being within a range between 30° C. and 60° C. for monitoring a growth status, so as to filter out initial cyanobacteria. Specifically, cultivation of the secondary mutant cyanobacteria can be carried out at an interval of 5° C. In this way, the initial cyanobacteria that are adapted to a growth environment of 30° C., 35° C., 40° C., 45° C., 50° C., 55° C., and 60° C. can be obtained.

In step S205, the initial cyanobacteria are placed in a mixed gas at a ventilation ratio within a range between 0.5% and 9%, so as to filter out target cyanobacteria. Specifically, the initial cyanobacteria that are adapted to a target temperature can be selected in step S204, and said initial cyanobacteria are placed in the mixed gas at a ventilation ratio of 0.5%, 1.0%, 1.5%, 2.0%, 2.5%, 3.0%, 5.0%, 7.0%, 9.0% for cultivation. The growth status thereof is monitored, so as to obtain the target cyanobacteria that are adapted to a specific ventilation condition.

In the present disclosure, the specific ventilation condition can be 30 ppm to 50 ppm of hydrogen, 150 ppm to 250 ppm of acetylene, 100 ppm to 200 ppm of methane, 0.1 ppm to 1 ppm of hydrogen sulfide, and 1 ppm to 5 ppm of acetaldehyde. Specifically, the specific ventilation condition can be 40 ppm of the hydrogen, 200 ppm of the acetylene, 150 ppm of the methane, 0.5 ppm of the hydrogen sulfide, and 3 ppm of the acetaldehyde. In addition, by subjecting the secondary mutant cyanobacteria to the chemical mutagenesis again, a growth temperature of the target cyanobacteria can be further increased. Hence, through the method for cultivating the cyanobacteria provided by the present disclosure, the probability of obtaining cyanobacteria that are capable of withstanding a high temperature and a high-temperature industrial waste gas can be effectively increased.

In order to illustrate that mixed mutagenesis (conducting UV mutagenesis before NTG mutagenesis) can increase the probability of obtaining a strain adapted to a high temperature, strains that are: (A) only subjected to the UV mutagenesis; (B) only subjected to the NTG mutagenesis; and (C) subjected to the mixed mutagenesis are picked and moved to a BG-11 liquid medium for high-temperature filtering and cultivation at different temperatures (in an environment where CO₂ is 1%). The growth status of these strains is monitored relative to time, such that the strain adapted to a high temperature can be obtained. Experiment results are shown in Table 2 below (the unit of temperature: ° C.).

TABLE 2

| | Growth status | | | Growth status | | | Growth status | |
|---|---|---|---|---|---|---|---|---|
| No. | Temperature | Status | No. | Temperature | Status | No. | Temperature | Status |
| A8 | 40 | ++ | B2 | 40 | +++ | C33 | 55 | ++ |
| A31 | 40 | ++ | B18 | 60 | + | C15 | 45 | +++ |
| A45 | 35 | +++ | B25 | 45 | +++ | C27 | 55 | ++ |
| A32 | 60 | + | B9 | 45 | ++ | C54 | 40 | +++ |
| A6 | 35 | +++ | B62 | 40 | ++ | C7 | 50 | + |
| A66 | 50 | ++ | B5 | 45 | +++ | C31 | 55 | + |

In Table 2, the growth status that is similar to or higher than a growth rate of an indigenous strain (a strain that is not subjected to mutagenesis) is deemed excellent (+++), the growth status that is lower than said growth rate by 0% to 25% is deemed good (++), and the growth status that is lower than said growth rate by 25% to 50% is deemed fair (+). Here, having a similar growth rate indicates that an amount of time required for obtaining an equivalent quantity of the strain is the same. In the present disclosure, the growth rate is the required amount of time for increasing from 0 to 1 based on OD₇₃₀ (optical density at 730 nm). Specific growth rate standards are shown in Table 3 below.

TABLE 3

| | | | Required time |
|---|---|---|---|
| | | | (minutes) for |
| | | | increasing |
| | Comparison | | from 0 to 1 |
| | result | Growth rate | based on OD₇₃₀ |
| Indigenous | — | — | 150 |
| strain | | | |
| Mutant | Excellent | similar to or higher | ≤150 |
| strain | (+++) | than that of the | |
| | | indigenous strain | |
| Mutant | Good (++) | lower by 0% to 25% | >150 to 187.5 |
| strain | | | |
| Mutant | Fair (+) | lower by 25% to 50% | >187.5 to 225 |
| strain | | | |
| Mutant | Poor (−) | lower by more than | >225 |
| strain | | 50% | |

According to the standards of Table 3, the growth status of the strain that is only processed by ultraviolet is evaluated to be excellent at 35° C., and is evaluated to be good at 40° C. The strain that is only processed by the NTG is capable of adapting to a temperature ranging between 40° C. and 45° C., but its growth status is evaluated to be fair when the temperature is raised to 60° C. However, the strain that is subjected to the mixed mutagenesis is capable of adapting to the temperature ranging between 40° C. and 45° C., and its growth status is also evaluated to be good at 55° C. As such, the results of Table 2 show that the strains that are subjected to the mixed mutagenesis have an improved growth status under a high temperature.

The strain that has an excellent or good growth status at a high temperature in the temperature resistance test (step S104) is further subjected to an environment resistance test (step S105). In the environment resistance test (step S105), the high-temperature-resistant strain is grown at a corresponding temperature, and a CO₂ mixed gas at the ventilation ratio within a range between 0.5% and 9% is used for testing. By monitoring the growth status of the strain relative to time, a strain adapted to growing at different concentrations of the CO₂ mixed gas can be obtained. Here, when the ventilation ratio is 1%, 1 mL of the CO₂ mixed gas is added into 1 L of culture volume per minute.

In the present disclosure, the CO₂ mixed gas is formulated based on a composition of an industrial waste gas that is to be applied. Specifically, the CO₂ mixed gas includes 40 ppm of hydrogen (H₂), 200 ppm of acetylene (C₂H₂), 150 ppm of methane (CH₄), 0.5 ppm of hydrogen sulfide (H₂S), and 3 ppm of acetaldehyde (CH₃CHO). Nos. A66, B25, B5, C33, C15, and C27 strains in Table 1 are subjected to the environment resistance test. Experiment results are shown in Table 4 below, and growth status evaluations are the same as those shown in Table 3.

TABLE 4

| | Growth status | | | Growth status | | | Growth status | |
|---|---|---|---|---|---|---|---|---|
| | Ventilation | | | Ventilation | | | Ventilation | |
| No. | ratio (%) | Status | No. | ratio (%) | Status | No. | ratio (%) | Status |
| C15 | 0.5 | +++ | B25 | 0.5 | ++ | B5 | 0.5 | +++ |
| 45° C. | 1.0 | +++ | 45° C. | 1.0 | + | 45° C. | 1.0 | + |
| | 1.5 | +++ | | 1.5 | + | | 1.5 | + |
| | 2.0 | ++ | | 2.0 | + | | 2.0 | + |
| | 2.5 | ++ | | 2.5 | + | | 2.5 | + |
| | 3.0 | ++ | | 3.0 | − | | 3.0 | + |
| | 5.0 | ++ | | 5.0 | − | | 5.0 | + |
| | 7.0 | + | | 7.0 | − | | 7.0 | + |
| | 9.0 | + | | 9.0 | − | | 9.0 | − |
| C33 | 0.5 | ++ | C27 | 0.5 | ++ | A66 | 0.5 | +++ |
| 55° C. | 1.0 | + | 55° C. | 1.0 | ++ | 50° C. | 1.0 | + |
| | 1.5 | + | | 1.5 | − | | 1.5 | + |
| | 2.0 | − | | 2.0 | − | | 2.0 | + |
| | 2.5 | − | | 2.5 | − | | 2.5 | − |
| | 3.0 | − | | 3.0 | − | | 3.0 | − |
| | 5.0 | − | | 5.0 | − | | 5.0 | − |
| | 7.0 | − | | 7.0 | − | | 7.0 | − |
| | 9.0 | − | | 9.0 | − | | 9.0 | − |

As shown in Table 4 above, the most exemplary example is No. C33 strain. At a high temperature of 55° C., No. C33 strain is still evaluated to have a good growth status when being placed in the CO2 mixed gas at the ventilation ratio of 1.5%. As such, No. C33 strain has characteristics of withstanding a high temperature and an industrial waste gas. Although No. A66 strain can withstand the CO₂ mixed gas up to the ventilation ratio of 2%, a growth temperature to which No. A66 strain is adapted is lower. In addition, out of the strains that are adapted to a temperature of 45° C., only No. C15 strain can withstand the CO₂ mixed gas up to the ventilation ratio of 9%. In order to increase the temperature that is endurable for No. C15 strain, No. C15 strain is further subjected to the NTG mutagenesis.

In one embodiment of the present disclosure, secondary NTG mutagenesis is to process the strain with 100 μg/ml of the NTG for 1 minute, such that the fatality rate is controlled to be approximately 50%. In one embodiment of the present disclosure, No. C15 strain is subjected to the secondary NTG mutagenesis, and the mutant strain is subjected to the temperature resistance test. Experiment results are shown in Table 5 below, and growth status evaluations are the same as those shown in Table 3.

TABLE 5

Growth status

| | Temperature | |
|---|---|---|
| No. | (° C.) | Status |
| C15-2 | 45 | ++ |
| C15-14 | 50 | ++ |
| C15-17 | 55 | +++ |
| C15-24 | 50 | +++ |
| C15-31 | 55 | +++ |
| C15-33 | 50 | + |
| C15-57 | 50 | ++ |
| C15-61 | 50 | + |
| C15-75 | 45 | ++ |
| C15-81 | 50 | ++ |
| C15-87 | 55 | +++ |
| C15-91 | 50 | + |

As shown in Table 5, the strain that has undergone the secondary NTG mutagenesis is capable of adapting to a temperature of 45° C. or more, and is particularly adapted to a temperature ranging between 50° C. and 55° C. Then, the strain that is evaluated to have an excellent growth status is further subjected to the environment resistance test. That is, Nos. C15-17, C15-31, and C15-87 strains in Table 5 are selected and subjected to the environment resistance test. Experiment results are shown in Table 6 below, and growth status evaluations are the same as those shown in Table 3.

TABLE 6

| | Growth status | | | Growth status | | | Growth status | |
|---|---|---|---|---|---|---|---|---|
| | Ventilation | | | Ventilation | | | Ventilation | |
| No. | ratio (%) | Status | No. | ratio (%) | Status | No. | ratio (%) | Status |
| C15- | 0.5 | +++ | C15- | 0.5 | ++ | C15- | 0.5 | +++ |
| 17 | 1.0 | +++ | 31 | 1.0 | ++ | 87 | 1.0 | ++ |
| 55° C. | 1.5 | +++ | 55° C. | 1.5 | ++ | 55° C. | 1.5 | ++ |
| | 2.0 | +++ | | 2.0 | + | | 2.0 | ++ |
| | 2.5 | +++ | | 2.5 | − | | 2.5 | ++ |
| | 3.0 | +++ | | 3.0 | − | | 3.0 | + |
| | 5.0 | +++ | | 5.0 | − | | 5.0 | + |
| | 7.0 | ++ | | 7.0 | − | | 7.0 | + |
| | 9.0 | ++ | | 9.0 | − | | 9.0 | − |

As shown in Table 6, the strain that has undergone the secondary NTG mutagenesis is capable of adapting to a temperature of 55° C., and its environment-resistant characteristic can also be improved. For example, No. C15-17 strain is capable of adapting to a temperature of 55° C. or more, and can still have a growth status similar to that of the indigenous strain in an environment where a ventilation concentration of the CO₂ mixed gas ranges between 0.5% and 5%. Furthermore, No. C15-17 strain can also have a good growth status in an environment where the CO₂ mixed gas has a high concentration (between 7% and 9%).

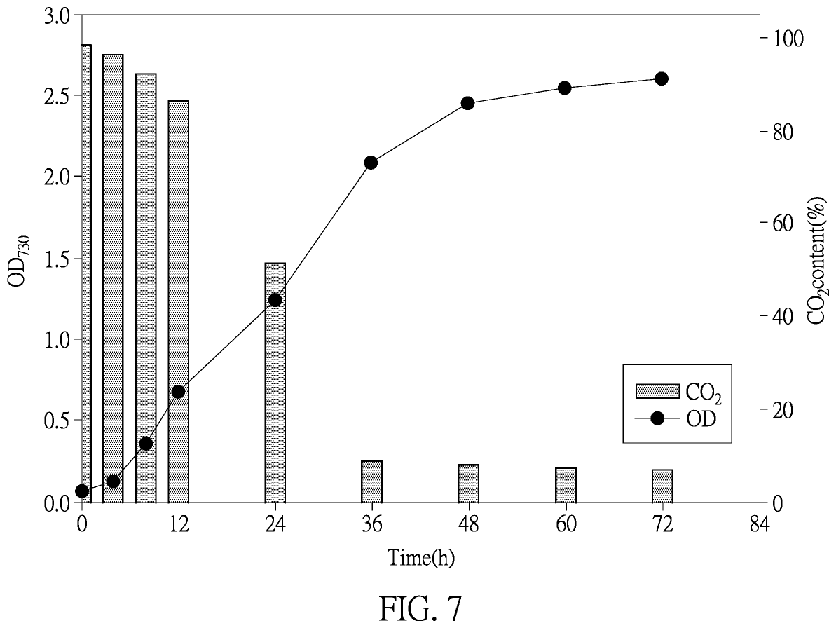

The strain (e.g., No. C15-17 strain) filtered out by the method of the present disclosure can be used to process the industrial waste gas. As shown in FIG. 7, the strain can still grow rapidly with time in the CO₂ mixed gas and at 45° C. In the first twelve hours, the quantity of the strain is low, such that the consumed CO₂ mixed gas is approximately 10%. However, with the rapid growth of the strain, the consumed CO₂ mixed gas is approximately 50% after twenty-four hours. After thirty-six hours, the consumed CO₂ mixed gas is approximately 90% or more. In other words, the strain filtered out by the method of the present disclosure can effectively process the CO₂ mixed gas, and can thus be used for processing the industrial waste gas.

### Beneficial Effects of the Embodiments

In conclusion, in the method for cultivating the cyanobacteria provided by the present disclosure, by virtue of “subjecting the cyanobacteria to the physical mutagenesis and the chemical mutagenesis” and “the fatality rate of the physical mutagenesis ranging between 50% and 80%, and the fatality rate of the chemical mutagenesis ranging between 40% and 55%,” the probability of obtaining cyanobacteria that are capable of withstanding a high-temperature industrial waste gas can be increased.

Furthermore, the present disclosure provides an effective mutagenesis process. By subjecting the secondary mutant cyanobacteria of the present disclosure to the NTG mutagenesis again, a growth temperature to which the cyanobacteria are adapted can be further increased, and a concentration of the industrial waste gas that is endurable for the cyanobacteria can also be increased. In this way, the cyanobacteria that are applicable for processing the high-temperature industrial waste gas can be more effectively obtained.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for cultivating cyanobacteria, comprising:
providing the cyanobacteria;
subjecting the cyanobacteria to physical mutagenesis, so as to obtain primary mutant cyanobacteria;
subjecting the primary mutant cyanobacteria to chemical mutagenesis, so as to obtain secondary mutant cyanobacteria; and
subjecting the secondary mutant cyanobacteria to a temperature resistance test and an environment resistance test, so as to obtain target cyanobacteria;
wherein a fatality rate of the physical mutagenesis ranges between 50% and 80%, and a fatality rate of the chemical mutagenesis ranges between 40% and 55%.

2. The method according to claim 1, wherein the fatality rate of the physical mutagenesis ranges between 60% and 80%.

3. The method according to claim 1, wherein the fatality rate of the chemical mutagenesis ranges between 45% and 55%.

4. The method according to claim 1, wherein, in the physical mutagenesis, an ultraviolet (UV) source having an illuminance of between 0.017 mW/cm² and 0.082 mW/cm² is used to irradiate the cyanobacteria for 10 seconds to 70 seconds.

5. The method according to claim 1, wherein nitrosoguanidine (NTG) having a concentration of between 50 μg/ml and 300 μg/ml is used in the chemical mutagenesis for a processing time within a range between 0.5 minutes and 2 minutes.

6. The method according to claim 1, wherein, in the temperature resistance test, the secondary mutant cyanobacteria are placed in an environment being within a range between 30° C. and 60° C. for monitoring a growth status, so as to filter out initial cyanobacteria.

7. The method according to claim 6, wherein, in the environment resistance test, the initial cyanobacteria are placed in a mixed gas at a ventilation ratio within a range between 0.5% and 9%, so as to filter out the target cyanobacteria.

8. The method according to claim 7, wherein the mixed gas includes hydrogen, acetylene, methane, hydrogen sulfide, and acetaldehyde.

9. The method according to claim 8, wherein the mixed gas includes 30 ppm to 50 ppm of the hydrogen, 150 ppm to 250 ppm of the acetylene, 100 ppm to 200 ppm of the methane, 0.1 ppm to 1 ppm of the hydrogen sulfide, and 1 ppm to 5 ppm of the acetaldehyde.

10. The method according to claim 1, further comprising:
subjecting the secondary mutant cyanobacteria to the chemical mutagenesis again.

11. A method for cultivating cyanobacteria, comprising:
providing the cyanobacteria;
subjecting the cyanobacteria to physical mutagenesis until a fatality rate reaches 50% to 80%, so as to obtain and use the cyanobacteria that survive the physical mutagenesis as primary mutant cyanobacteria;
subjecting the primary mutant cyanobacteria to chemical mutagenesis until the fatality rate reaches 40% to 55%, so as to obtain and use the primary mutant cyanobacteria that survive the chemical mutagenesis as secondary mutant cyanobacteria;
placing the secondary mutant cyanobacteria in an environment being within a range between 30° C. and 60° C. for monitoring a growth status, so as to filter out initial cyanobacteria; and
placing the initial cyanobacteria in a mixed gas at a ventilation ratio within a range between 0.5% and 9%, so as to filter out target cyanobacteria;
wherein the mixed gas is a mixture that includes hydrogen, acetylene, methane, hydrogen sulfide, and acetaldehyde.

12. The method according to claim 11, wherein, in the physical mutagenesis, an ultraviolet (UV) source having an illuminance of between 0.017 mW/cm² and 0.082 mW/cm² is used to irradiate the cyanobacteria for 10 seconds to 70 seconds.

13. The method according to claim 11, wherein nitrosoguanidine (NTG) having a concentration of between 50 μg/ml and 300 μg/ml is used in the chemical mutagenesis for a processing time within a range between 0.5 minutes and 2 minutes.

14. The method according to claim 11, wherein the mixed gas includes 30 ppm to 50 ppm of the hydrogen, 150 ppm to 250 ppm of the acetylene, 100 ppm to 200 ppm of the methane, 0.1 ppm to 1 ppm of the hydrogen sulfide, and 1 ppm to 5 ppm of the acetaldehyde.

15. The method according to claim 11, further comprising:
subjecting the secondary mutant cyanobacteria to the chemical mutagenesis again.
